(19) United States
(12) Patent Application Publication
(10) Pub. No.: US 2005/0148935 A1
(43) Pub. Date: Jul. 7, 2005

(54) Botulinum toxin injection guide

(76) Inventors: Rozalina Dimitrova, Mission Viejo, CA (US); Vandenburgh Amanda, Huntington Beach, CA (US); Walcott M. Jennifer, Laguna Niguel, CA (US)

Correspondence Address: STEPHEN DONOVAN;ALLERGAN, INC., 2525 Dupont Drive, T2-7H, Irvine, CA, 92612, US

(21) Appl. No.: 10/748,999

(22) Filed: Dec. 29, 2003

(51) Int. Cl. A61M 5/00

(52) U.S. Cl. 604/116

(57) ABSTRACT

An injection guide to assist a botulinum toxin therapy is disclosed. The injection guide can comprise a flexible, plastic sheet with staggered holes for placement over an area of skin at which a patient experiences pain. After placement of the injection guide a pen or other mark is inserted through the holes to thereby easily establish a visible grid of where to inject a botulinum toxin.

### BACKGROUND

The present invention relates to a device for assisting a botulinum toxin therapy. In particular, the present invention relates to an injection guide for assisting botulinum toxin therapy.

Subcutaneous and intramuscular administration of a botulinum toxin is known to treat various diseases. Typically, a syringe or a needleless device is used to inject the botulinum toxin to the dermal or subdermal target tissue. For some diseases, such as neuralgia, multiple injections of the botulinum toxin can be required over a relatively small area of the skin. Multiple injections are carried out to achieve a desired distribution and therapeutic diffusion of the botulinum toxin into the target area, as opposed to making only one or a few injections. Typically, after determining the dimensions or parameters of a dermal area into which a botulinum toxin administration is to be carried out (i.e. into a dermal or subdermal area from which the patient reports or experiences pain), the physician or his assistant attempts to indicate the locations of botulinum toxin injection by manually (i.e. freehand) marking a pattern (a grid) of multiple spaced dots onto the target skin area. Often the injection location dots are neither properly spaced nor of an appropriate number when such a freehand injection grid marking method is used. It is known to use a multiple injection plate for the treatment of hyperhydrosis wherein five or seven needles puncture the skin at the same time. Grimalt R., et al., Multi-injection plate for botulinum toxin application in the treatment of axillary hyperhidrosis, Dermatol Surg 2001 June; 27(6): 543-544.

Botulinum Toxin

The genus Clostridium has more than one hundred and twenty seven species, grouped according to their morphology and functions. The anaerobic, gram positive bacterium Clostridium botulinum produces a potent polypeptide neurotoxin, botulinum toxin, which causes a neuroparalytic illness in humans and animals referred to as botulism. The spores of Clostridium botulinum are found in soil and can grow in improperly sterilized and sealed food containers of home based canneries, which are the cause of many of the cases of botulism. The effects of botulism typically appear 18 to 36 hours after eating the foodstuffs infected with a Clostridium botulinum culture or spores. The botulinum toxin can apparently pass unattenuated through the lining of the gut and attack peripheral motor neurons. Symptoms of botulinum toxin intoxication can progress from difficulty walking, swallowing, and speaking to paralysis of the respiratory muscles and death.

Botulinum toxin type A is the most lethal natural biological agent known to man. About 50 picograms of a commercially available botulinum toxin type A (purified neurotoxin complex)¹ is a LD₅₀ in mice (i.e. 1 unit). One unit of BOTOX® contains about 50 picograms (about 56 attomoles) of botulinum toxin type A complex. Interestingly, on a molar basis, botulinum toxin type A is about 1.8 billion times more lethal than diphtheria, about 600 million times more lethal than sodium cyanide, about 30 million times more lethal than cobra toxin and about 12 million times more lethal than cholera. Singh, Critical Aspects of Bacterial Protein Toxins, pages 63-84 (chapter 4) of Natural Toxins II, edited by B. R. Singh et al., Plenum Press, New York (1976) (where the Available from Allergan, Inc., of Irvine, Calif. under the tradename BOTOX® in 100 unit vials) stated LD₅₀ of botulinum toxin type A of 0.3 ng equals 1 U is corrected for the fact that about 0.05 ng of BOTOX® equals 1 unit). One unit (U) of botulinum toxin is defined as the LD₅₀ upon intraperitoneal injection into female Swiss Webster mice weighing 18 to 20 grams each.

Seven generally immunologically distinct botulinum neurotoxins have been characterized, these being respectively botulinum neurotoxin serotypes A, B, C₁, D, E, F and G each of which is distinguished by neutralization with type-specific antibodies. The different serotypes of botulinum toxin vary in the animal species that they affect and in the severity and duration of the paralysis they evoke. For example, it has been determined that botulinum toxin type A is 500 times more potent, as measured by the rate of paralysis produced in the rat, than is botulinum toxin type B. Additionally, botulinum toxin type B has been determined to be non-toxic in primates at a dose of 480 U/kg which is about 12 times the primate LD₅₀ for botulinum toxin type A. Moyer E et al., Botulinum Toxin Type B: Experimental and Clinical Experience, being chapter 6, pages 71-85 of “Therapy With Botulinum Toxin”, edited by Jankovic, J. et al. (1994), Marcel Dekker, Inc. Botulinum toxin apparently binds with high affinity to cholinergic motor neurons, is translocated into the neuron and blocks the release of acetylcholine. Additional uptake can take place through low affinity receptors, as well as by phagocytosis and pinocytosis.

Regardless of serotype, the molecular mechanism of toxin intoxication appears to be similar and to involve at least three steps or stages. In the first step of the process, the toxin binds to the presynaptic membrane of the target neuron through a specific interaction between the heavy chain, H chain, and a cell surface receptor; the receptor is thought to be different for each type of botulinum toxin and for tetanus toxin. The carboxyl end segment of the H chain, Hc, appears to be important for targeting of the toxin to the cell surface.

In the second step, the toxin crosses the plasma membrane of the poisoned cell. The toxin is first engulfed by the cell through receptor-mediated endocytosis, and an endosome containing the toxin is formed. The toxin then escapes the endosome into the cytoplasm of the cell. This step is thought to be mediated by the amino end segment of the H chain, H_{N}, which triggers a conformational change of the toxin in response to a pH of about 5.5 or lower. Endosomes are known to possess a proton pump which decreases intra-endosomal pH. The conformational shift exposes hydrophobic residues in the toxin, which permits the toxin to embed itself in the endosomal membrane. The toxin (or at a minimum the light chain) then translocates through the endosomal membrane into the cytoplasm.

The last step of the mechanism of botulinum toxin activity appears to involve reduction of the disulfide bond joining the heavy chain, H chain, and the light chain, L chain. The entire toxic activity of botulinum and tetanus toxins is contained in the L chain of the holotoxin; the L chain is a zinc (Zn++) endopeptidase which selectively cleaves proteins essential for recognition and docking of neurotransmitter-containing vesicles with the cytoplasmic surface of the plasma membrane, and fusion of the vesicles with the plasma membrane. Tetanus neurotoxin, botulinum toxin types B, D, F, and G cause degradation of synaptobrevin (also called vesicle-associated membrane protein (VAMP)), a synaptosomal membrane protein. Most of the VAMP present at the cytoplasmic surface of the synaptic vesicle is removed as a result of any one of these cleavage events. Botulinum toxin serotype A and E cleave SNAP-25. Botulinum toxin serotype C₁ was originally thought to cleave syntaxin, but was found to cleave syntaxin and SNAP-25. Each of the botulinum toxins specifically cleaves a different bond, except botulinum toxin type B (and tetanus toxin) which cleave the same bond. Each of these cleavages block the process of vesicle-membrane docking, thereby preventing exocytosis of vesicle content.

Although all the botulinum toxins serotypes apparently inhibit release of the neurotransmitter acetylcholine at the neuromuscular junction, they do so by affecting different neurosecretory proteins and/or cleaving these proteins at different sites. For example, botulinum types A and E both cleave the 25 kiloDalton (kD) synaptosomal associated protein (SNAP-25), but they target different amino acid sequences within this protein. Botulinum toxin types B, D, F and G act on vesicle-associated protein (VAMP, also called synaptobrevin), with each serotype cleaving the protein at a different site. Finally, botulinum toxin type C₁ has been shown to cleave both syntaxin and SNAP-25. These differences in mechanism of action may affect the relative potency and/or duration of action of the various botulinum toxin serotypes. Apparently, a substrate for a botulinum toxin can be found in a variety of different cell types. See e.g. Biochem J 1; 339 (pt 1): 159-65: 1999, and Mov Disord, 10(3):376: 1995 (pancreatic islet B cells contains at least SNAP-25 and synaptobrevin).

The molecular weight of the botulinum toxin protein molecule, for all seven of the known botulinum toxin serotypes, is about 150 kD. Interestingly, the botulinum toxins are released by Clostridial bacterium as complexes comprising the 150 kD botulinum toxin protein molecule along with associated non-toxin proteins. Thus, the botulinum toxin type A complex can be produced by Clostridial bacterium as 900 kD, 500 kD and 300 kD forms. Botulinum toxin types B and C₁ is apparently produced as only a 700 kD or 500 kD complex. Botulinum toxin type D is produced as both 300 kD and 500 kD complexes. Finally, botulinum toxin types E and F are produced as only approximately 300 kD complexes. The complexes (i.e. molecular weight greater than about 150 kD) are believed to contain a non-toxin hemaglutinin protein and a non-toxin and non-toxic nonhemaglutinin protein. These two non-toxin proteins (which along with the botulinum toxin molecule comprise the relevant neurotoxin complex) may act to provide stability against denaturation to the botulinum toxin molecule and protection against digestive acids when toxin is ingested. Additionally, it is possible that the larger (greater than about 150 kD molecular weight) botulinum toxin complexes may result in a slower rate of diffusion of the botulinum toxin away from a site of intramuscular injection of a botulinum toxin complex.

In vitro studies have indicated that botulinum toxin inhibits potassium cation induced release of both acetylcholine and norepinephrine from primary cell cultures of brainstem tissue. Additionally, it has been reported that botulinum toxin inhibits the evoked release of both glycine and glutamate in primary cultures of spinal cord neurons and that in brain synaptosome preparations botulinum toxin inhibits the release of each of the neurotransmitters acetylcholine, dopamine, norepinephrine (Habermann E., et al., Tetanus Toxin and Botulinum A and C Neurotoxins Inhibit Noradrenaline Release From Cultured Mouse Brain, J Neurochem 51(2); 522-527: 1988) CGRP, substance P and glutamate (Sanchez-Prieto, J., et al., Botulinum Toxin A Blocks Glutamate Exocytosis From Guinea Pig Cerebral Cortical Synaptosomes, Eur J. Biochem 165; 675-681: 1897. Thus, when adequate concentrations are used, stimulus-evoked release of most neurotransmitters is blocked by botulinum toxin. See e.g. Pearce, L. B., Pharmacologic Characterization of Botulinum Toxin For Basic Science and Medicine, Toxicon 35(9); 1373-1412 at 1393; Bigalke H., et al., Botulinum A Neurotoxin Inhibits Non-Cholinergic Synaptic Transmission in Mouse Spinal Cord Neurons in Culture, Brain Research 360; 318-324: 1985; Habermann E., Inhibition by Tetanus and Botulinum A Toxin of the release of [³H]Noradrenaline and [³H]GABA From Rat Brain Homogenate, Experientia 44; 224-226: 1988, Bigalke H., et al., Tetanus Toxin and Botulinum A Toxin Inhibit Release and Uptake of Various Transmitters, as Studied with Particulate Preparations From Rat Brain and Spinal Cord, Naunyn-Schmiedeberg's Arch Pharmacol 316; 244-251: 1981, and; Jankovic J. et al., Therapy With Botulinum Toxin, Marcel Dekker, Inc., (1994), page 5.

Botulinum toxin type A can be obtained by establishing and growing cultures of Clostridium botulinum in a fermenter and then harvesting and purifying the fermented mixture in accordance with known procedures. All the botulinum toxin serotypes are initially synthesized as inactive single chain proteins which must be cleaved or nicked by proteases to become neuroactive. The bacterial strains that make botulinum toxin serotypes A and G possess endogenous proteases and serotypes A and G can therefore be recovered from bacterial cultures in predominantly their active form. In contrast, botulinum toxin serotypes C₁, D and E are synthesized by nonproteolytic strains and are therefore typically unactivated when recovered from culture. Serotypes B and F are produced by both proteolytic and nonproteolytic strains and therefore can be recovered in either the active or inactive form. However, even the proteolytic strains that produce, for example, the botulinum toxin type B serotype only cleave a portion of the toxin produced. The exact proportion of nicked to unnicked molecules depends on the length of incubation and the temperature of the culture. Therefore, a certain percentage of any preparation of, for example, the botulinum toxin type B toxin is likely to be inactive, possibly accounting for the known significantly lower potency of botulinum toxin type B as compared to botulinum toxin type A. The presence of inactive botulinum toxin molecules in a clinical preparation will contribute to the overall protein load of the preparation, which has been linked to increased antigenicity, without contributing to its clinical efficacy. Additionally, it is known that botulinum toxin type B has, upon intramuscular injection, a shorter duration of activity and is also less potent than botulinum toxin type A at the same dose level.

High quality crystalline botulinum toxin type A can be produced from the Hall A strain of Clostridium botulinum with characteristics of >3×10⁷ U/mg, an A₂₆₀/A₂₇₈ of less than 0.60 and a distinct pattern of banding on gel electrophoresis. The known Shantz process can be used to obtain crystalline botulinum toxin type A, as set forth in Shantz, E. J., et al, Properties and use of Botulinum toxin and Other Microbial Neurotoxins in Medicine, Microbiol Rev. 56; 80-99: 1992. Generally, the botulinum toxin type A complex can be isolated and purified from an anaerobic 10 fermentation by cultivating Clostridium botulinum type A in a suitable medium. The known process can also be used, upon separation out of the non-toxin proteins, to obtain pure botulinum toxins, such as for example: purified botulinum toxin type A with an approximately 150 kD molecular weight with a specific potency of 1-2×10⁸ LD₅₀ U/mg or greater; purified botulinum toxin type B with an approximately 156 kD molecular weight with a specific potency of 1-2×10⁸ LD₅₀ U/mg or greater, and; purified botulinum toxin type F with an approximately 155 kD molecular weight with a specific potency of 1-2×10⁷ LD₅₀ U/mg or greater.

Botulinum toxins and/or botulinum toxin complexes can be obtained from List Biological Laboratories, Inc., Campbell, Calif.; the Centre for Applied Microbiology and Research, Porton Down, U.K.; Wako (Osaka, Japan), Metabiologics (Madison, Wis.) as well as from Sigma Chemicals of St Louis, Mo. Pure botulinum toxin can also be used to prepare a pharmaceutical composition.

As with enzymes generally, the biological activities of the botulinum toxins (which are intracellular peptidases) is dependant, at least in part, upon their three dimensional conformation. Thus, botulinum toxin type A is detoxified by heat, various chemicals surface stretching and surface drying. Additionally, it is known that dilution of the toxin complex obtained by the known culturing, fermentation and purification to the much, much lower toxin concentrations used for pharmaceutical composition formulation results in rapid detoxification of the toxin unless a suitable stabilizing agent is present. Dilution of the toxin from milligram quantities to a solution containing nanograms per milliliter presents significant difficulties because of the rapid loss of specific toxicity upon such great dilution. Since the toxin may be used months or years after the toxin containing pharmaceutical composition is formulated, the toxin can stabilized with a stabilizing agent such as albumin and gelatin.

The success of botulinum toxin type A to treat a variety of clinical conditions has led to interest in other botulinum toxin serotypes. Two commercially available botulinum type A preparations for use in humans are BOTOX® available from Allergan, Inc., of Irvine, Calif., and Dysport® available from Beaufour Ipsen, Porton Down, England. A Botulinum toxin type B preparation (MyoBloc®) is available from Elan Pharmaceuticals of San Francisco, Calif.

BOTOX® consists of a purified botulinum toxin type A complex, albumin and sodium chloride packaged in sterile, vacuum-dried form. The botulinum toxin type A is made from a culture of the Hall strain of Clostridium botulinum grown in a medium containing N-Z amine and yeast extract. The botulinum toxin type A complex is purified from the culture solution by a series of acid precipitations to a crystalline complex consisting of the active high molecular weight toxin protein and an associated hemagglutinin protein. The crystalline complex is re-dissolved in a solution containing saline and albumin and sterile filtered (0.2 microns) prior to vacuum-drying. The vacuum-dried product is stored in a freezer at or below −5° C. BOTOX® can be reconstituted with sterile, non-preserved saline prior to intramuscular injection. Each vial of BOTOX® contains about 100 units (U) of Clostridium botulinum toxin type A purified neurotoxin complex, 0.5 milligrams of human serum albumin and 0.9 milligrams of sodium chloride in a sterile, vacuum-dried form without a preservative.

To reconstitute vacuum-dried BOTOX®, sterile normal saline without a preservative; (0.9% Sodium Chloride Injection) is used by drawing up the proper amount of diluent in the appropriate size syringe. Since BOTOX® may be denatured by bubbling or similar violent agitation, the diluent is gently injected into the vial. For sterility reasons BOTOX® is preferably administered within four hours after the vial is removed from the freezer and reconstituted. During these four hours, reconstituted BOTOX® can be stored in a refrigerator at about 2° C. to about 8° C. Reconstituted, refrigerated BOTOX® has been reported to retain its potency for at least about two weeks. Neurology, 48: 249-53: 1997.

Botulinum toxins have been used in clinical settings for the treatment of neuromuscular disorders characterized by hyperactive skeletal muscles (i.e. motor disorders). In 1989 a botulinum toxin type A complex has been approved by the U.S. Food and Drug Administration for the treatment of blepharospasm, strabismus and hemifacial spasm. Subsequently, a botulinum toxin type A was also approved by the FDA for the treatment of cervical dystonia and for the treatment of glabellar lines, and a botulinum toxin type B was approved for the treatment of cervical dystonia. Non-type A botulinum toxin serotypes apparently have a lower potency and/or a shorter duration of activity as compared to botulinum toxin type A. Clinical effects of peripheral intramuscular botulinum toxin type A are usually seen within one week of injection. The typical duration of symptomatic relief from a single intramuscular injection of botulinum toxin type A averages about three months, although significantly longer periods of therapeutic activity have been reported.

It has been reported that botulinum toxin type A has been used in clinical settings as follows:
(1) about 75-125 units of BOTOX® per intramuscular injection (multiple muscles) to treat cervical dystonia;
(2) 5-10 units of BOTOX® per intramuscular injection to treat glabellar lines (brow furrows) (5 units injected intramuscularly into the procerus muscle and 10 units injected intramuscularly into each corrugator supercilii muscle);
(3) about 30-80 units of BOTOX® to treat constipation by intrasphincter injection of the puborectalis muscle;
(4) about 1-5 units per muscle of intramuscularly injected BOTOX® to treat blepharospasm by injecting the lateral pre-tarsal orbicularis oculi muscle of the upper lid and the lateral pre-tarsal orbicularis oculi of the lower lid.
(5) to treat strabismus, extraocular muscles have been injected intramuscularly with between about 1-5 units of BOTOX®, the amount injected varying based upon both the size of the muscle to be injected and the extent of muscle paralysis desired (i.e. amount of diopter correction desired).
(6) to treat upper limb spasticity following stroke by intramuscular injections of BOTOX® into five different upper limb flexor muscles, as follows:
(a) flexor digitorum profundus: 7.5 U to 30 U
(b) flexor digitorum sublimus: 7.5 U to 30 U
(c) flexor carpi ulnaris: 10 U to 40 U
(d) flexor carpi radialis: 15 U to 60 U
(e) biceps brachii: 50 U to 200 U. Each of the five indicated muscles has been injected at the same treatment session, so that the patient receives from 90 U to 360 U of upper limb flexor muscle BOTOX® by intramuscular injection at each treatment session.

(7) to treat migraine, pericranial injected (injected symmetrically into glabellar, frontalis and temporalis muscles) injection of 25 U of BOTOX® has showed significant benefit as a prophylactic treatment of migraine compared to vehicle as measured by decreased measures of migraine frequency, maximal severity, associated vomiting and acute medication use over the three month period following the 25 U injection.
(8) to treat neuropathic pain syndromes such as complex regional pain syndrome (CRPS) by injecting 300 U of BOTOX® into the sternocleidomastoid, trapezius, splenius capitis, splenius cervicis, levator scapular, supraspinatus, infraspinatus, or rhomboid major muscle groups. See e.g. Argoff, A Focused Review on the Use of Botulinum Toxins for Neuropathic Pain, Clin J Pain 18(6 Suppl); S177-S181: 2002.
(9) to treat cervical spinal cord injuries with multiple subcutaneous injections (about 16-20) of 5 U (a total dose approximately of 100 U) of BOTOX®. Ibid.
(10) to treat postherpetic neuralgia (PHN) using 5 U of BOTOX® per 0.1 ml of normal saline for every 9 cm of painful skin (total doses did not exceed 200 U). Ibid.

It is known that botulinum toxin type A can have an efficacy for up to 12 months (European J. Neurology 6 (Supp 4): S11-S1150: 1999), and in some circumstances for as long as 27 months, when used to treat glands, such as in the treatment of hyperhydrosis. See e.g. Bushara K., Botulinum toxin and rhinorrhea, Otolaryngol Head Neck Surg 1996; 114(3): 507, and The Laryngoscope 109: 1344-1346: 1999. However, the usual duration of an intramuscular injection of Botox® is typically about 3 to 4 months.

In addition to having pharmacologic actions at the peripheral location, botulinum toxins may also have inhibitory effects in the central nervous system. Work by Weigand et al, Nauny-Schmiedeberg's Arch. Pharmacol. 1976; 292, 161-165, and Habermann, Nauny-Schmiedeberg's Arch. Pharmacol. 1974; 281, 47-56 showed that botulinum toxin is able to ascend to the spinal area by retrograde transport. As such, a botulinum toxin injected at a peripheral location, for example intramuscularly, may be retrograde transported to the spinal cord.

A botulinum toxin has also been proposed for the treatment of rhinorrhea (chronic discharge from the nasal mucous membranes, i.e. runny nose), rhinitis (inflammation of the nasal mucous membranes), hyperhydrosis and other disorders mediated by the autonomic nervous system (U.S. Pat. No. 5,766,605), tension headache, (U.S. Pat. No. 6,458,365), migraine headache (U.S. Pat. No. 5,714,468), post-operative pain and visceral pain (U.S. Pat. No. 6,464,986), pain treatment by intraspinal toxin administration (U.S. Pat. No. 6,113,915), Parkinson's disease and other diseases with a motor disorder component, by intracranial toxin administration (U.S. Pat. No. 6,306,403), hair growth and hair retention (U.S. Pat. No. 6,299,893), psoriasis and dermatitis (U.S. Pat. No. 5,670,484), injured muscles (U.S. Pat. No. 6,423,319, various cancers (U.S. Pat. No. 6,139,845), pancreatic disorders (U.S. Pat. No. 6,143,306), smooth muscle disorders (U.S. Pat. No. 5,437,291, including injection of a botulinum toxin into the upper and lower esophageal, pyloric and anal sphincters)), prostate disorders (U.S. Pat. No. 6,365,164), inflammation, arthritis and gout (U.S. Pat. No. 6,063,768), juvenile cerebral palsy (U.S. Pat. No. 6,395,277), inner ear disorders (U.S. Pat. No. 6,265,379), thyroid disorders (U.S. Pat. No. 6,358,513), parathyroid disorders (U.S. Pat. No. 6,328,977) and neurogenic inflammation (U.S. Pat. No. 6,063,768). Additionally, controlled release toxin implants are known (see e.g. U.S. Pat. Nos. 6,306,423 and 6,312,708).

U.S. patent application Ser. No. 10/621,054, filed Jul. 15, 2003, entitled “Device to assist hyperhydrosis therapy” sets forth a dermal overlay device for assisting hyperhydrosis therapy. This device does not permit staggered injection sites to be indicated on a patient's skin.

Tetanus toxin, as wells as derivatives (i.e. with a non-native targeting moiety), fragments, hybrids and chimeras thereof can also have therapeutic utility. The tetanus toxin bears many similarities to the botulinum toxins. Thus, both the tetanus toxin and the botulinum toxins are polypeptides made by closely related species of Clostridium (Clostridium tetani and Clostridium botulinum, respectively). Additionally, both the tetanus toxin and the botulinum toxins are dichain proteins composed of a light chain (molecular weight about 50 kD) covalently bound by a single disulfide bond to a heavy chain (molecular weight about 100 kD). Hence, the molecular weight of tetanus toxin and of each of the seven botulinum toxins (non-complexed) is about 150 kD. Furthermore, for both the tetanus toxin and the botulinum toxins, the light chain bears the domain which exhibits intracellular biological (protease) activity, while the heavy chain comprises the receptor binding (immunogenic) and cell membrane translocational domains.

Further, both the tetanus toxin and the botulinum toxins exhibit a high, specific affinity for gangliocide receptors on the surface of presynaptic cholinergic neurons. Receptor mediated endocytosis of tetanus toxin by peripheral cholinergic neurons results in retrograde axonal transport, blocking of the release of inhibitory neurotransmitters from central synapses and a spastic paralysis. Contrarily, receptor mediated endocytosis of botulinum toxin by peripheral cholinergic neurons results in little if any retrograde transport, inhibition of acetylcholine exocytosis from the intoxicated peripheral motor neurons and a flaccid paralysis.

Finally, the tetanus toxin and the botulinum toxins resemble each other in both biosynthesis and molecular architecture. Thus, there is an overall 34% identity between the protein sequences of tetanus toxin and botulinum toxin type A, and a sequence identity as high as 62% for some functional domains. Binz T. et al., The Complete Sequence of Botulinum Neurotoxin Type A and Comparison with Other Clostridial Neurotoxins, J Biological Chemistry 265(16); 9153-9158: 1990.

Acetylcholine

Typically only a single type of small molecule neurotransmitter is released by each type of neuron in the mammalian nervous system, although there is evidence which suggests that several neuromodulators can be released by the same neuron. The neurotransmitter acetylcholine is secreted by neurons in many areas of the brain, but specifically by the large pyramidal cells of the motor cortex, by several different neurons in the basal ganglia, by the motor neurons that innervate the skeletal muscles, by the preganglionic neurons of the autonomic nervous system (both sympathetic and parasympathetic), by the bag 1 fibers of the muscle spindle fiber, by the postganglionic neurons of the parasympathetic nervous system, and by some of the postganglionic neurons of the sympathetic nervous system. Essentially, only the postganglionic sympathetic nerve fibers to the sweat glands, the piloerector muscles and a few blood vessels are cholinergic as most of the postganglionic neurons of the sympathetic nervous system secret the neurotransmitter norepinephine. In most instances acetylcholine has an excitatory effect. However, acetylcholine is known to have inhibitory effects at some of the peripheral parasympathetic nerve endings, such as inhibition of heart rate by the vagal nerve.

The efferent signals of the autonomic nervous system are transmitted to the body through either the sympathetic nervous system or the parasympathetic nervous system. The preganglionic neurons of the sympathetic nervous system extend from preganglionic sympathetic neuron cell bodies located in the intermediolateral horn of the spinal cord. The preganglionic sympathetic nerve fibers, extending from the cell body, synapse with postganglionic neurons located in either a paravertebral sympathetic ganglion or in a prevertebral ganglion. Since, the preganglionic neurons of both the sympathetic and parasympathetic nervous system are cholinergic, application of acetylcholine to the ganglia will excite both sympathetic and parasympathetic postganglionic neurons.

Acetylcholine activates two types of receptors, muscarinic and nicotinic receptors. The muscarinic receptors are found in all effector cells stimulated by the postganglionic, neurons of the parasympathetic nervous system as well as in those stimulated by the postganglionic cholinergic neurons of the sympathetic nervous system. The nicotinic receptors are found in the adrenal medulla, as well as within the autonomic ganglia, that is on the cell surface of the postganglionic neuron at the synapse between the preganglionic and postganglionic neurons of both the sympathetic and parasympathetic systems. Nicotinic receptors are also found in many nonautonomic nerve endings, for example in the membranes of skeletal muscle fibers at the neuromuscular junction.

Acetylcholine is released from cholinergic neurons when small, clear, intracellular vesicles fuse with the presynaptic neuronal cell membrane. A wide variety of non-neuronal secretory cells, such as, adrenal medulla (as well as the PC12 cell line) and pancreatic islet cells release catecholamines and parathyroid hormone, respectively, from large dense-core vesicles. The PC12 cell line is a clone of rat pheochromocytoma cells extensively used as a tissue culture model for studies of sympathoadrenal development. Botulinum toxin inhibits the release of both types of compounds from both types of cells in vitro, permeabilized (as by electroporation) or by direct injection of the toxin into the denervated cell. Botulinum toxin is also known to block release of the neurotransmitter glutamate from cortical synaptosomes cell cultures.

A neuromuscular junction is formed in skeletal muscle by the proximity of axons to muscle cells. A signal transmitted through the nervous system results in an action potential at the terminal axon, with activation of ion channels and resulting release of the neurotransmitter acetylcholine from intraneuronal synaptic vesicles, for example at the motor endplate of the neuromuscular junction. The acetylcholine crosses the extracellular space to bind with acetylcholine receptor proteins on the surface of the muscle end plate. Once sufficient binding has occurred, an action potential of the muscle cell causes specific membrane ion channel changes, resulting in muscle cell contraction. The acetylcholine is then released from the muscle cells and metabolized by cholinesterases in the extracellular space. The metabolites are recycled back into the terminal axon for reprocessing into further acetylcholine.

Antinociceptive Properties and Mechanism of Action

Botulinum toxin can affect neurons within the CNS. For example, botulinum toxin serotypes B and F and tetanus toxin are internalized by cultured rat hippocampal astrocytes and cleave the appropriate substrate. Neuropeptide release was reported to be inhibited by botulinum toxin (botulinum toxins A, B, C1, F) treatment in vitro from embryonic rat dorsal root ganglia neurons and from isolated rabbit iris sphincter and dilatory muscles. More importantly, the in vitro release of acetylcholine and substance P (but not norepinephrine) from the rabbit ocular tissue was also inhibited with botulinum toxin A. Therefore, based on these in vitro and limited in vivo data, it can be hypothesized that botulinum toxin treatment may reduce the local release of nociceptive neuropeptides from either cholinergic neurons or from C or A delta fibers in vivo. The reduced neuropeptide release could prevent the local sensitization of nociceptors and thus reduce the perception of pain. A reduction of nociceptive signals from the periphery could then reduce the central sensitization associated with chronic pain. This effect on the nociceptive neurons could work in concert with the other well-known effects of botulinum toxin on the cholinergic motor neuron innervating the extrafusal and intrafusal fibers.

Botulinum toxin therapy has been reported to alleviate pain associated with various conditions with or without concomitant excess muscle contractions. Aoki K. R., Pharmacology and immunology of botulinum toxin serotypes, J Neurol 248(suppl 1); I/3-I/10: 2001. Early observations in patients with cervical dystonia who were treated with BOTOX® suggested that the pain relief exceeded the motor benefit. In other areas, the pain associated with myoclonus of spinal cord origin has been treated effectively with BOTOX®. Tension-associated headaches have been reported to be alleviated with BOTOX® therapy. In a double-blind placebo-controlled trial, investigators reported profound antinociceptive activity of intramuscular BOTOX® when administered prior to aductor-release surgery in children with cerebral palsy. The effect was so dramatic that the trial was terminated early. Children treated with BOTOX® had a reduced need for narcotic analgesics, were discharged earlier, and had better outcomes than the placebo group. In a pilot study, patients with chronic whiplash-associated neck pain were successfully treated with BOTOX®. Other reports of BOTOX® for reduction of primary pain include trigger point injections, myofascial pain and migraine headache prophylaxis, and back pain.

A preclinical investigation on the local antinociceptive efficacy of BOTOX® has been reported. A rat model of inflammatory pain was used to demonstrate that a subcutaneous injection of BOTOX® prevented the classical behavioral pain response to a subplantar injection of formalin. Cui M, Aoki KR, Botulinum toxin type a (BTX-a) reduced inflammatory pain in the rat formalin model, Cephalalgia 20(4); 414: 2000. BOTOX® (3.5 and 7 units/kg) was administered subcutaneously to the plantar surface of the rat 5 days before the formalin challenge in the same area. BOTOX® produced local antinociceptive effects without obvious muscle weakness.

BOTOX® has also been shown to dose dependently inhibit formalin-induced glutamate release in the rat paw and the expression of C-fos in the dorsal horn of the spinal cord. Cui M, Li Z, You S, Khanijou S, Aoki KR, Mechanisms of Antinociceptive Effect of Subcutaneous BOTOX®: Inhibition of Peripheral and Central Nociceptive Processing, Naunyn Schmiedegergs Arch Pharmacol 265(Suppl 2); R17: 2002. BOTO® has also been shown to inhibit calcitonin gene-related peptide (CGRP) release from trigeminal ganglia nerves. Durham P, Cady R, Cady R, Mechanism of botulinum toxin type-A Inhibition of Calcitonin Gene-Related Peptide Secretion from Trigeminal Nerve Cells, Cephalalgia 23(7); 690: 2003. Using microdialysis, it was found that BOTOX® inhibited capsaicin-induced thermal hyperalgesia suggesting an action on substance P. Aoki K R, Cui M, Mechanisms of the Antinociceptive Effect of Subcutaneous BOTOX®: Inhibition of Peripheral and Central Nociceptive Processing, Cephalalgia 23(7); 649: 2003. These results indicate that subcutaneous BOTOX® inhibits neurotransmitter release from primary sensory neurons in the rat formalin model. Through this mechanism, BOTOX® inhibits peripheral sensitization in these models, which leads to an indirect reduction in central sensitization.

The preclinical (in vitro and in vivo) evidence coupled with the clinical observations strongly suggests that botulinum toxin (especially BOTOX®) may have a separate antinociceptive effect from its well-known effect on the neuromuscular junction and other cholinergic nerves.

What is needed therefore is an injection guide for facilitating botulinum toxin therapy by assisting the marking of a target skin area with a grid or pattern of staggered injection location marks or dots, at which locations (i.e. at the dots) a botulinum toxin can be injected.

### SUMMARY

The present invention meets this need and provides an injection guide for facilitating botulinum toxin therapy by assisting the marking of a target skin area with a grid or pattern of staggered injection location marks or dots, at which locations (i.e. at the dots) a botulinum toxin can be injected.

The botulinum toxin (as either a complex [i.e. about 300 to about 900 kDa] or as a pure [i.e. about 150 kDa molecule]) used can be a botulinum toxin type A, B, C, D, E, F or G.

As used herein “about” means approximately or nearly and in the context of a numerical value or range set forth herein means±10% of the numerical value or range recited or claimed.

An injection guide for assisting botulinum toxin therapy according to our invention can comprise a material with an upper face and a lower face. The lower face of the material is suitable for placement in contact with an area of the dermis of a patient. The dermal area can be an area at which the patient experiences pain. The material can have a plurality of staggered perforations which extend completely through the material from the upper face to the lower face. By “staggered” it is meant that if a straight line A is drawn through all the perforations of any one row of perforations comprising the injection guide, then a line B drawn at a right angle to any one of the perforations upon the line A will not intersect any perforation present in a row of perforations immediately adjacent to (i.e. in the row above or in the row below the line A row) the perforations on the line A.

Additionally, the material can have an exterior border which circumscribes the material. The exterior border is not perforated because a user presses down on the border to hold the device in place when it is in use.

Preferably, the material is flexible, so that when the material is pressed again the dermal area, substantially all of the exterior border is in contact with the dermal area. The perforations in the material can be spaced apart in a staggered pattern.

A method for assisting a botulinum toxin therapy through use of our injection guide can have the steps of: determining a dermal area of a patient to which an administration of a botulinum toxin is required; placing in contact with the dermal area the lower face of the injection guide comprising; extending a marker through a perforation so as to mark a dermal surface under the lower face of the material, and; removing the device from contact with the dermal area.

This method can further comprise after the removing step, the step of injecting a botulinum toxin at the location of each mark or dot placed on the dermal area.

### DRAWINGS

The following drawings are provided to assist understanding of aspects and features of the present invention.

FIG. 1 is top view of an embodiment of an injection guide for assisting a botulinum toxin therapy within the scope of the present invention, showing a plurality of perforations in the device.

FIG. 2 is a is top view of a second embodiment of an injection guide for assisting a botulinum toxin therapy within the scope of the present invention, showing a plurality of more closely set together perforations, as compared to the FIG. 1 embodiment.

FIG. 3 is a is top view of a third embodiment of an injection guide for assisting a botulinum toxin therapy within the scope of the present invention, showing a plurality of perforations, which are more widely spaced apart as compared to the FIG. 2 embodiment, and fewer in number as compared to the FIG. 1 embodiment.

### DESCRIPTION

Our invention is based upon our discovery of an injection guide that can be used to assist a botulinum toxin therapy.

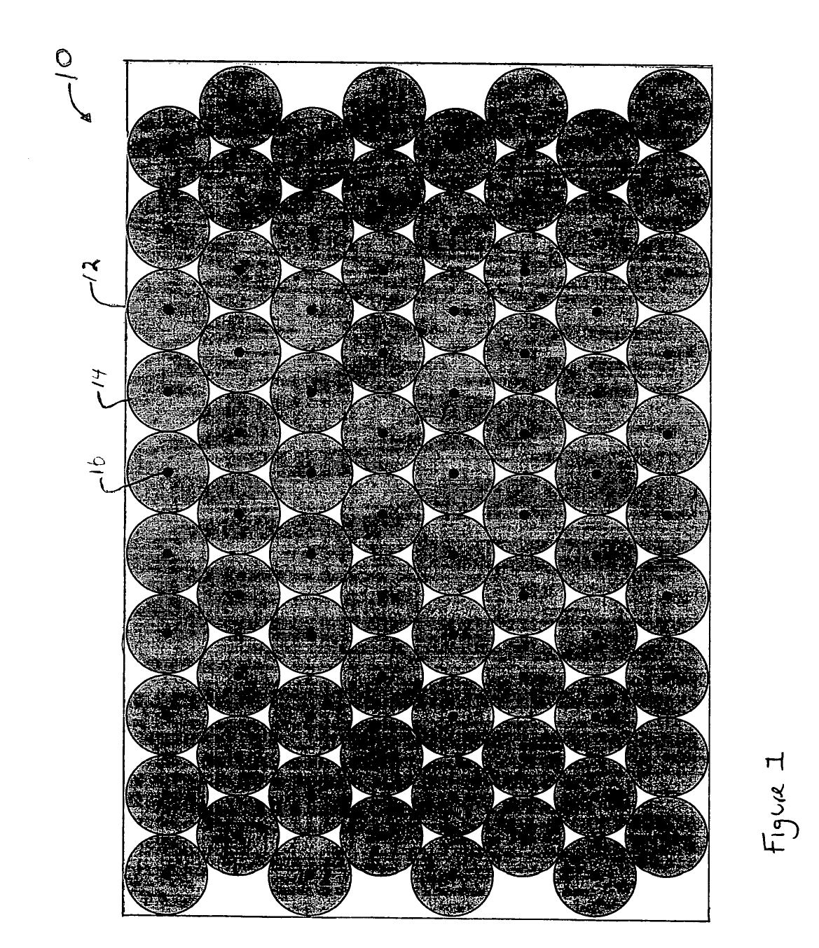

An embodiment within the scope of our invention is shown by FIG. 1. An injection guide 10 can comprise a flat, transparent material 12 upon which are can be rendered a number (eighty in FIG. 1) of contiguous circles 14. Contiguous circles 14 upon the material 12 are used as a convenient means to space apart perforations 16. Other suitable means or geometric structures for spacing the perforations 16 can be used. The material 12 can be a flexible material with a flat surface, such as a transparent plastic sheet. The circles 14 can be drawn, printed or etched on the material 12. The center of each circle 12 can have a perforation 16 which extends through the material 14. The perforation or hole 16 is sized to permit the tip of a marker, such as a felt tip pen to be passed there through. Thus, upon placement of the material in contact with the skin of a patient to be treated with a intradermal injection of a botulinum toxin, use of the marker as indicated above and then removal of the material 12 from contact with the surface of the skin of the patient, one obtains thereby a grid of marked points on the surface of the skin of the patient, indicating where the botulinum toxin should be injected (subcutaneous or intramuscular toxin injection). Use of the FIG. 1 embodiment can permit a grid to be established for 80 injections of i.e. 2.5 units of a botulinum toxin at each marked injection site, for a total therefore of a 200 unit botulinum toxin injection. The circles are sized in the FIG. 1 embodiment so as to space each injection site 2 cm apart.

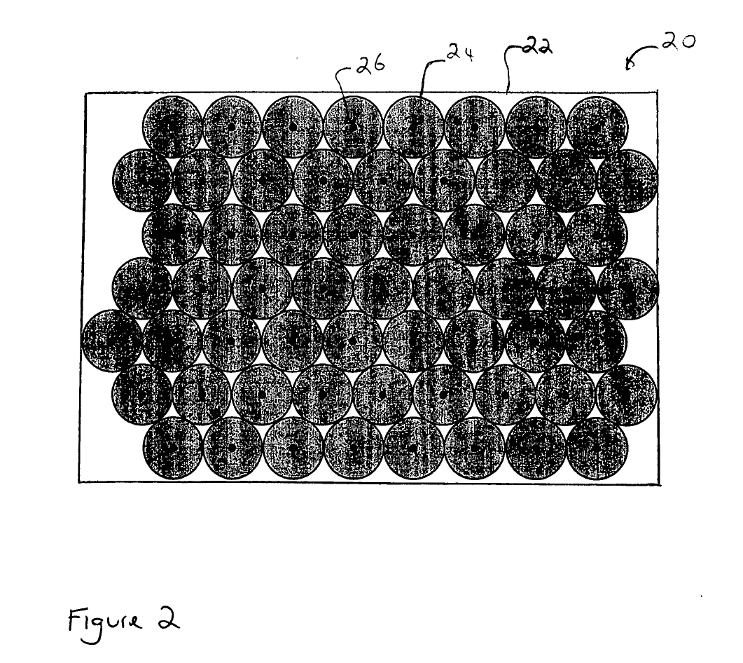

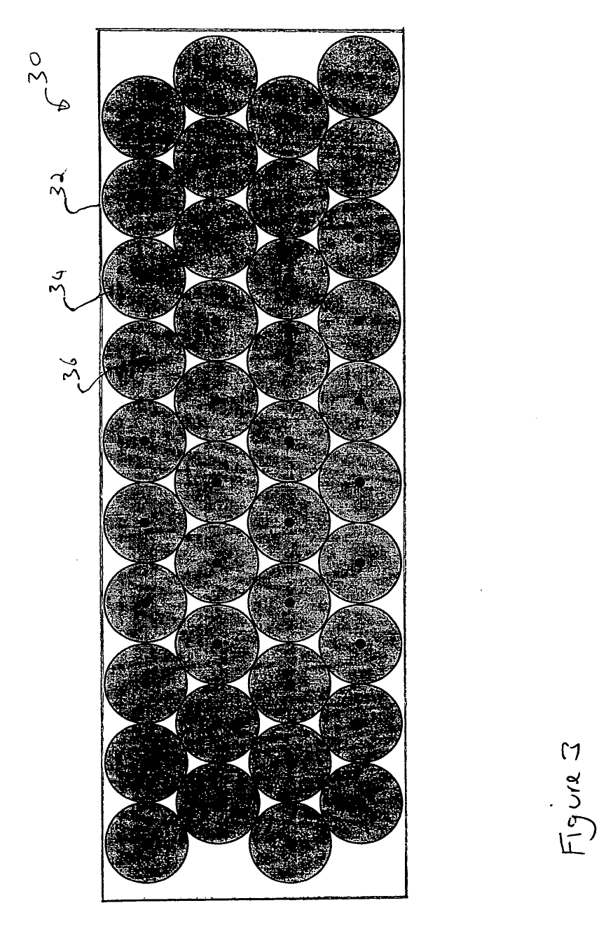

An alternate embodiment of our invention is shown by FIG. 2. As shown by FIG. 2, an injection guide 20 can comprise a flat, transparent material 22 upon which are rendered a number (sixty one in FIG. 1) of contiguous circles 24. The material 22 can again be a flexible material with a flat surface, such as a transparent plastic sheet. The circles 24 can be drawn, printed or etched on the material 22. The center of each circle 24 can have a perforation 26 which extends through the material 24. The FIG. 2 embodiment can permit 61 times 2.5 units or 152.2 units of a botulinum toxin injection. The circles are sized in the FIG. 2 embodiment so as to space each injection site 1.5 cm apart. Numerous embodiments with different numbers of centrally perforated circles are within the scope of our invention. For example the FIG. 3 shows an embodiment of our invention which comprises an injection guide 30 comprised of a flat, transparent material 32 upon which are rendered a number (forty in FIG. 3) of contiguous circles 34. The material 32 can again be a flexible material with a flat surface, such as a transparent plastic sheet and the center of each circle 34 can have a perforation 36 which extends through the material 34. The FIG. 3 embodiment has forty perforated circles, so that if five units of a botulinum toxin are injected at each forty dermal sites marked by using the FIG. 3 embodiment (in which embodiment the perforated circles are spaced 1.5 cm apart), then 200 units of a botulinum toxin will be injected.

Injection of a botulinum toxin to treat many different indications can be facilitated through use of our invention, such as use to facilitate treatment of postherpetic neuralgia.

It is important to note that all the embodiments of our invention comprise rows of staggered circles so as to provide a grid of evenly spaced toxin injection points. Thus, use of an injection guide with linear (non-staggered) circles would provide a grid where all the injection points are not equidistant from one another other. Hence upon injection of toxin with a grid established by use of an injection guide with linear rows of circles, a uniform toxin diffusion will not be obtained.

Using a clear, flexible material allows for the delineating of the areas of injection onto the guide. The area of pain and/or allodynia are first marked on the patient using a surgical grade marker. The guide is then placed over the patient and the same areas traced onto the guide. The injection sites are then marked on the patient. The size of the surface area of the marked injection guide can then be easily determined using planimetry.

Each embodiment of our invention can be comprised of a material, such as a flexible plastic, suitable (i.e. no sharp protrusions, non-irritating) for firm, though temporary, placement against a patch or area of the skin of a patient.

In practice the injection guide can be used by placing the lower face of the guide against an area of target skin. The injection guide is pressed against the skin and a marker is inserted into each of the perforations in turn. The device in then removed form contact with the skin leaving a grid pattern of dots on the skin showing where to inject the botulinum toxin.

The material which comprises the device can be a plastic, silicone or other suitable material. The material can be flexible and can be shaped and sized so as to follow the contours of an armpit, foot or hand where it can be applied.

Examples of botulinum toxins within the scope of the present invention include the botulinum toxin types A, B, C, D, E, F, and G.

Botulinum toxins for use according to the present invention can be stored in lyophilized, vacuum dried form in containers under vacuum pressure or as stable liquids. Prior to lyophilization the botulinum toxin can be combined with pharmaceutically acceptable excipients, stabilizers and/or carriers, such as albumin. The lyophilized material can be reconstituted with saline or water to create a solution or composition containing the botulinum toxin to be administered to the patient.

Our invention includes a method for determining an area of pain and/or allodynia by placing in contact with the dermal area of a patient the lower face of the injection guide and extending a probe through a perforation into contact with the underlying dermal area and then recording or noting the patient's response as to an absence or a presence of pain and/or allodynia upon the probe contact occurring. The probe extending step is then repeated as many times as necessary until the extent of the dermal area at which the patient experiences pain and/or allodynia has been determined. The probe can be any suitable item such as a cotton swab or covered pen tip.

Additionally, our invention also includes a method for determining an area of pain and/or allodynia by first mapping out an area of pain and/or allodynia on the skin of a patient (i.e. by using a cotton swap to touch the patient's skin and noting his or her response) followed by placing in contact with the dermal area of a patient a lower face of the injection guide. There can then be drawn upon the upper surface of the guide (i.e. with a marker or felt pen) the boundary (i.e. the parameters of the area, as a circle drawn) of the area of pain and/or allodynia experienced by the patient. The area within the boundary circle drawn on the upper surface of the material (as measured by planimetry, for example) thereby determines the dermal area of pain and/or allodynia.

### EXAMPLES

The following non-limiting examples set forth specific preferred methods to use a device within the scope of the present invention and are not intended to limit the scope of the invention.

### Example 1

### Use of an Injection Guide to Facilitate Treatment of Post Hepatic Neuralgia

A patient presents with postherpetic neuralgia. The patient can be treated by administering 5 units to 800 units of a botulinum toxin type A (i.e. 2.5U per injection site, 3 to 80 injections). Areas of pain and allodynia can be delineated on the patient using a surgical marker. An injection guide can be placed on the subject and the areas of pain and allodynia can be marked through use of the injection guide. If the area of pain and allodynia covers 2 or fewer circles on a 2 cm injection guide, then a 1.5 cm injection guide can be used. The injections sites can be marked on the patient using the appropriate 2 cm or 1.5 cm injection guide. The injection guides can then be evaluated with a planimeter to determine the surface area for the areas of pain and allodynia. The doctor can use the injection guide without marking the injection sites, but only marking the areas of pain or allodynia. If the guides are utilized in this manner, this will assist the doctor in determining if the areas of pain or allodynia change over a specified period of time.

### Example 1

### Use of Device for Assisting Pain Therapy

A female patient, 32 years old, reports chronic pain on her arm. The lower side of the injection guide of in FIG. 1 is pressed firmly against area of skin where she reports the sensation of pain and a suitable marker is inserted into each of the perforations of the injection guide in turn. The areas of pain and allodynia are also drawn on the injection guide (e.g., pain using a solid line and allodynia using a dotted line). The injection guide is removed, leaving a clear (and temporary) grid pattern of dots on her arm. A botulinum toxin can then be injected at the site of each dot, thereby treating her pain. Using a planimeter, it can be determined that the area of pain is 25 cm² and the area of allodynia is 50 cm². One month later, the patient can return to see the doctor. Using the injection guide, the doctor can again traces the areas of pain and allodynia on the injection guide. Using planimetry, it can be determined that the area of pain is now 10 cm² and the area of allodynia is now 35 cm². The doctor can utilize this information to make the determination that the injection are working. The doctor can decide to administer another treatment to the patient. Using the holes in the injection guide, the injection sites are marked on the patient. The injection guide is removed and the injections are administered to the patient.

Although the present invention has been described in detail with regard to certain preferred methods, other embodiments, versions, and modifications within the scope of the present invention are possible. For example, the disclosed device can be made from various materials and in various shapes, with different perforations spacings and different perforation bore diameters.

All references, articles, patents, applications and publications set forth above are incorporated herein by reference in their entireties.

Accordingly, the spirit and scope of the following claims should not be limited to the descriptions of the preferred embodiments set forth above.

1. An injection guide for assisting administration of a botulinum toxin, the injection guide comprising:
(a) a material having an upper face and a lower face, the lower face of the material being suitable for placement in contact with an area of the dermis of a patient to or through which dermal area a botulinum toxin can be administered, and;
(b) the material having a plurality of staggered perforations which extend completely through the material from the upper face to the lower face.

2. The injection guide of claim 1, wherein the material has an exterior border which circumscribes the material and wherein the exterior border is not perforated.

3. The injection guide of claim 2, wherein the material is flexible, so that when the material is pressed again the dermal area, substantially all of the exterior border is in contact with the dermal area.

4. The injection guide of claim 1 wherein at least some of the perforations are spaced apart by a uniform distance.

5. A injection guide for assisting a botulinum toxin therapy, the injection guide comprising:
(a) a material with an upper face and a lower face, the lower face of the material being suitable for placement in contact with an area of the dermis of a patient;
(b) the material has a plurality of staggered perforations which extend completely through the material from the upper face to the lower face, and;
(c) the material is flexible, so that when the material is pressed again the dermal area, substantially all of the lower face of the material is in contact with the dermal area.

6. The injection guide of claim 5 wherein at least some of the perforations are spaced apart by a uniform distance.

7. The injection guide of claim 5, wherein the material comprises a plurality of circles.

8. The injection guide of claim 7, wherein the material comprises a plurality of contiguous circles.

9. The injection guide of claim 8, wherein the perforations are located in the center of the circles.

10. A injection guide for assisting a botulinum toxin therapy, the injection guide comprising:
(a) a material with an upper face and a lower face, the lower face of the material being suitable for placement in contact with an area of the dermis of a patient;
(b) the material has a plurality of staggered perforations which extend completely through the material from the upper face to the lower face, wherein at least some of the perforations are spaced apart by a uniform distance;
(c) the material is flexible, so that when the material is pressed again the dermal area, substantially all of the lower face of the material is in contact with the dermal area, and;
(d) a plurality of contiguous circles are rendered upon the material and the perforations are located in the center of the circles.

11. A method for assisting a botulinum toxin therapy, the method comprising the steps of:
(a) determining a dermal area of a patient for administration of a botulinum toxin;
(b) placing in contact with the dermal area a lower face of a injection guide comprising: (i) a material with an upper face and a lower face, and; (ii) the material having a plurality of staggered perforations which extend completely through the material from the upper face to the lower face;
(c) extending a marker through a perforation so as to mark a dermal surface under the lower face of the material, and;
(d) removing the injection guide from contact with the dermal area.

12. The method of claim 11, further comprising after the removing step, the step of injecting a botulinum toxin at the location of a mark on the dermal area.

11. A method for determining an area of pain and/or allodynia, the method comprising the steps of:
(a) placing in contact with the dermal area of a patient a lower face of a injection guide comprising: (i) a material with an upper face and a lower face, and; (ii) the material having a plurality of staggered perforations which extend completely through the material from the upper face to the lower face;
(c) extending a probe through a perforation into contact with the underlying dermal area and recording the patient's response as to an absence or a presence of pain and/or allodynia upon the contact occurring, and;
(d) repeating step (c) to thereby determine the extent of the dermal area at which the patient experiences pain and/or allodynia.

12. A method for determining an area of pain and/or allodynia, the method comprising the steps of:
(a) placing in contact with the dermal area of a patient a lower face of a injection guide comprising: (i) a material with an upper face and a lower face, and; (ii) the material having a plurality of staggered perforations which extend completely through the material from the upper face to the lower face;
(c) drawing upon the upper face of the material a boundary of a dermal area at which the patient experiences pain and/or allodynia, and;
(d) determining the area within the boundary, thereby determining the area of pain and/or allodynia.
